(19) United States
(12) Patent Application Publication
(10) Pub. No.: US 2026/0061185 A1
(43) Pub. Date: Mar. 5, 2026

(54) COCHLEAR IMPLANT ASSEMBLIES, ELECTRODE LEADS, AND METHODS OF MANUFACTURING THE SAME

(71) Applicant: Advanced Bionics LLC, Valencia, CA (US)

(72) Inventors: Nick Apollo, Valencia, CA (US); Anil K. Patnala, Stevenson Ranch, CA (US); Anthony J. Spahr, Newhall, CA (US); R. Tissa Karunasiri, Valencia, CA (US); Jason Galster, Studio City, CA (US)

(21) Appl. No.: 19/105,689

(22) PCT Filed: Aug. 24, 2022
(86) PCT No.: PCT/US2022/041378

§ 371 Date: Feb. 21, 2025

(51) Int. Cl. A61N 1/05; A61N 1/36

(52) U.S. Cl. CPC A61N 1/0541; A61N 1/0551; A61N 1/36038; A61N 1/3605

(57) ABSTRACT

An exemplary electrode lead includes a plurality of metallic electrode contacts and an elastomeric encapsulant that encapsulates the plurality of metallic electrode contacts and that defines an outer surface of the electrode lead. The elastomeric encapsulant includes a non-conductive portion configured to define a portion of the outer surface of the electrode lead and a plurality of conductive portions that form a remainder of the outer surface of the electrode lead. Each of the conductive portions included in the plurality of conductive portions is in conductive contact with a respective metallic electrode contact included in the plurality of metallic electrode contacts.

### BACKGROUND INFORMATION

Cochlear implant systems are used to provide, restore, and/or improve the sense of hearing to recipients with severe or profound hearing loss. Conventional cochlear implant systems include various components configured to be implanted within a recipient (e.g., an electronics package, an antenna, and an electrode lead) and various components configured to be located external to the recipient (e.g., a sound processor, a battery, and a microphone). Typically, at least some of the implanted components of a cochlear implant system are provided within an encapsulant formed of a biocompatible material such as medical grade silicone.

An electrode lead of a cochlear implant system includes an electrode array comprised of metal contacts (e.g., platinum, titanium, etc.) insulated by the medical grade silicone. To create an electrochemical interface for recording and/or stimulation, the metal contacts in conventional electrode leads are typically exposed to target tissue. However, such exposure subjects the metal/silicone interfaces along the electrode array to harsh conditions of body fluids that may include chloride salts, reactive oxygen species, enzymes, etc. In addition, it is challenging to create robust, hermetic bonds between the medical grade silicone and the noble metal contacts due to the chemical incompatibility between the materials. As a result, the metal/silicone interfaces may deteriorate over time, which could potentially lead to leakage pathways into the electrode lead, feedthroughs, and the possibly electronics package.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system.

FIG. 2 shows an exemplary configuration of the cochlear implant system of FIG. 1.

FIG. 3 shows another exemplary configuration of the cochlear implant system of FIG. 1.

FIG. 4 shows an exemplary cochlear implant assembly according to principles described herein.

FIG. 5 shows an exemplary electrode lead according to principles described herein.

FIG. 6 is an exemplary cross section of the electrode lead shown in FIG. 6 taken along lines 6-6 in FIG. 6 according to principles described herein.

FIG. 7 is an exemplary cross section of the electrode lead shown in FIG. 6 taken along lines 7-7 in FIG. 6 according to principles described herein.

FIGS. 8-9 show additional exemplary electrode leads according to principles described herein.

FIG. 10 shows an exemplary method for manufacturing an electrode lead according to principles described herein.

### DETAILED DESCRIPTION

Cochlear implant assemblies, electrode leads, and methods for manufacturing the same are described herein. An exemplary cochlear implant assembly described herein includes an electrode lead and an implantable cochlear stimulator (ICS) configured to apply electrical stimulation to the recipient by way of the electrode lead. The electrode lead includes a plurality of metallic electrode contacts and an elastomeric encapsulant that encapsulates the plurality of metallic electrode contacts and that defines an outer surface of the electrode lead. The elastomeric encapsulant includes a non-conductive portion configured to define a portion of the outer surface of the electrode lead and a plurality of conductive portions that form a remainder of the outer surface of the electrode lead. Each of the conductive portions included in the plurality of conductive portions is in conductive contact with a respective metallic electrode contact included in the plurality of metallic electrode contacts.

An exemplary electrode lead includes a plurality of metallic electrode contacts and an elastomeric encapsulant that encapsulates the plurality of metallic electrode contacts and that defines an outer surface of the electrode lead. The elastomeric encapsulant includes a non-conductive portion configured to define a portion of the outer surface of the electrode lead and a plurality of conductive portions that form a remainder of the outer surface of the electrode lead. Each of the conductive portions included in the plurality of conductive portions is in conductive contact with a respective metallic electrode contact included in the plurality of metallic electrode contacts.

The cochlear implant assemblies and electrode leads described herein may provide various benefits to cochlear implant recipients, as well as others involved with managing cochlear implant systems. For example, the electrode leads such as those described herein do not include encapsulants having a metal electrode and elastomer interface that is in contact with tissue and/or body fluid. As a result, electrode leads such as those described herein are relatively insusceptible to leakage, corrosion, and/or device failure due to the chemical compatibility of the insulating and conducting elastomers. In addition, because such electrode leads are relatively insusceptible to leakage, they are relatively easy to manage by clinicians, which may result in fewer updates to a stimulation protocol that may otherwise arise due to leakage or failure at the metal electrode and elastomer interface. Accordingly, cochlear implant systems that include electrode leads such as those described herein are robust and can have a relatively long operational life.

Moreover, electrode leads such as those described herein may include a ground electrode (also referred to as an indifferent electrode (“IE”)) that is relatively larger than a conventional ground electrode of an electrode lead and that has improved features. For example, the increased size of the ground electrode results in a reduction of impedance at the ground. This may reduce error during perioperative impedance measurements. Further, by using a conductive elastomer rather than a relatively larger rigid (e.g., metallic) electrode at a tissue contact surface, it is possible to reduce a mechanical mismatch and thus strain at an interface between the conductive portion and the non-conducive portion of an elastomeric encapsulant.

Various embodiments will now be described in more detail with reference to the figures. The disclosed cochlear implant assemblies, electrode leads, and methods may provide one or more of the benefits mentioned above and/or various additional and/or alternative benefits that will be made apparent herein.

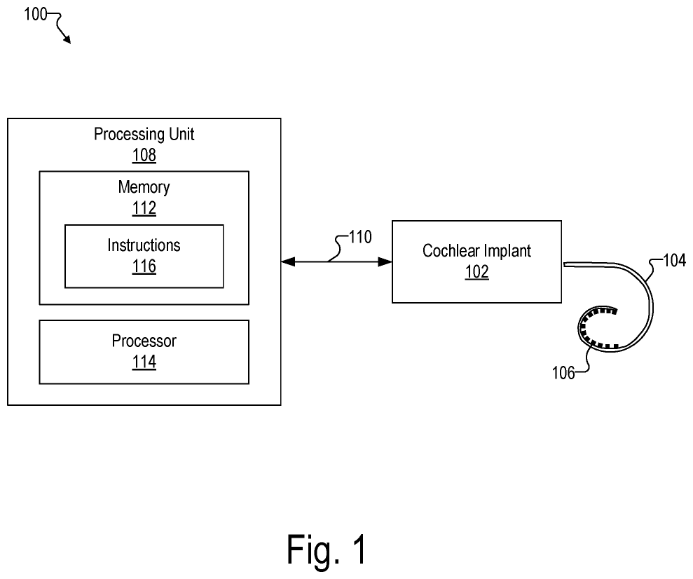

FIG. 1 illustrates an exemplary cochlear implant system 100 configured to be used by a recipient. As shown, cochlear implant system 100 includes a cochlear implant 102, an electrode lead 104 physically coupled to cochlear implant 102 and having an array of electrodes 106, and a processing unit 108 configured to be communicatively coupled to cochlear implant 102 by way of a communication link 110.

The cochlear implant system 100 shown in FIG. 1 is unilateral (i.e., associated with only one ear of the recipient). Alternatively, a bilateral configuration of cochlear implant system 100 may include separate cochlear implants and electrode leads for each ear of the recipient. In the bilateral configuration, processing unit 108 may be implemented by a single processing unit configured to interface with both cochlear implants or by two separate processing units each configured to interface with a different one of the cochlear implants.

Cochlear implant 102 may be implemented by any suitable type of implantable stimulator. For example, cochlear implant 102 may be implemented by an implantable cochlear stimulator. Additionally or alternatively, cochlear implant 102 may be implemented by a brainstem implant and/or any other type of device that may be implanted within the recipient and configured to apply electrical stimulation to one or more stimulation sites located along an auditory pathway of the recipient.

In some examples, cochlear implant 102 may be configured to generate electrical stimulation representative of an audio signal processed by processing unit 108 in accordance with one or more stimulation parameters transmitted to cochlear implant 102 by processing unit 108. Cochlear implant 102 may be further configured to apply the electrical stimulation to one or more stimulation sites (e.g., one or more intracochlear locations) within the recipient by way of one or more electrodes 106 on electrode lead 104. In some examples, cochlear implant 102 may include a plurality of independent current sources each associated with a channel defined by one or more of electrodes 106. In this manner, different stimulation current levels may be applied to multiple stimulation sites simultaneously by way of multiple electrodes 106.

Cochlear implant 102 may additionally or alternatively be configured to generate, store, and/or transmit data. For example, cochlear implant may use one or more electrodes 106 to record one or more signals (e.g., one or more voltages, impedances, evoked responses within the recipient, and/or other measurements) and transmit, by way of communication link 110, data representative of the one or more signals to processing unit 108. In some examples, this data is referred to as back telemetry data.

Electrode lead 104 may be implemented in any suitable manner. For example, a distal portion of electrode lead 104 may be pre-curved such that electrode lead 104 conforms with the helical shape of the cochlea after being implanted. Electrode lead 104 may alternatively be naturally straight or of any other suitable configuration.

In some examples, electrode lead 104 includes a plurality of wires (e.g., within an outer sheath) that conductively couple electrodes 106 to one or more current sources within cochlear implant 102. For example, if there are n electrodes 106 on electrode lead 104 and n current sources within cochlear implant 102, there may be n separate wires within electrode lead 104 that are configured to conductively connect each electrode 106 to a different one of the n current sources. Exemplary values for n are 8, 12, 16, or any other suitable number.

Electrodes 106 are located on at least a distal portion of electrode lead 104. In this configuration, after the distal portion of electrode lead 104 is inserted into the cochlea, electrical stimulation may be applied by way of one or more of electrodes 106 to one or more intracochlear locations. One or more other electrodes (e.g., including a ground electrode, not explicitly shown) may also be disposed on other parts of electrode lead 104 (e.g., on a proximal portion of electrode lead 104) to, for example, provide a current return path for stimulation current applied by electrodes 106 and to remain external to the cochlea after the distal portion of electrode lead 104 is inserted into the cochlea. Additionally or alternatively, a housing of cochlear implant 102 may serve as a ground electrode for stimulation current applied by electrodes 106.

Processing unit 108 may be configured to interface with (e.g., control and/or receive data from) cochlear implant 102. For example, processing unit 108 may transmit commands (e.g., stimulation parameters and/or other types of operating parameters in the form of data words included in a forward telemetry sequence) to cochlear implant 102 by way of communication link 110. Processing unit 108 may additionally or alternatively provide operating power to cochlear implant 102 by transmitting one or more power signals to cochlear implant 102 by way of communication link 110. Processing unit 108 may additionally or alternatively receive data from cochlear implant 102 by way of communication link 110. Communication link 110 may be implemented by any suitable number of wired and/or wireless bidirectional and/or unidirectional links.

As shown, processing unit 108 includes a memory 112 and a processor 114 configured to be selectively and communicatively coupled to one another. In some examples, memory 112 and processor 114 may be distributed between multiple devices and/or multiple locations as may serve a particular implementation.

Memory 112 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (“RAM”), and an optical disc. Exemplary volatile storage media include, but are not limited to, RAM (e.g., dynamic RAM).

Memory 112 may maintain (e.g., store) executable data used by processor 114 to perform one or more of the operations described herein. For example, memory 112 may store instructions 116 that may be executed by processor 114 to perform any of the operations described herein. Instructions 116 may be implemented by any suitable application, program (e.g., sound processing program), software, code, and/or other executable data instance. Memory 112 may also maintain any data received, generated, managed, used, and/or transmitted by processor 114.

Processor 114 may be configured to perform (e.g., execute instructions 116 stored in memory 112 to perform) various operations with respect to cochlear implant 102.

To illustrate, processor 114 may be configured to control an operation of cochlear implant 102. For example, processor 114 may receive an audio signal (e.g., by way of a microphone communicatively coupled to processing unit 108, a wireless interface (e.g., a Bluetooth interface), and/or a wired interface (e.g., an auxiliary input port)). Processor 114 may process the audio signal in accordance with a sound processing program (e.g., a sound processing program stored in memory 112) to generate appropriate stimulation parameters. Processor 114 may then transmit the stimulation parameters to cochlear implant 102 to direct cochlear implant 102 to apply electrical stimulation representative of the audio signal to the recipient.

In some implementations, processor 114 may also be configured to apply acoustic stimulation to the recipient. For example, a receiver (also referred to as a loudspeaker) may be optionally coupled to processing unit 108. In this configuration, processor 114 may deliver acoustic stimulation to the recipient by way of the receiver. The acoustic stimulation may be representative of an audio signal (e.g., an amplified version of the audio signal), configured to elicit an evoked response within the recipient, and/or otherwise configured. In configurations in which processor 114 is configured to both deliver acoustic stimulation to the recipient and direct cochlear implant 102 to apply electrical stimulation to the recipient, cochlear implant system 100 may be referred to as a bimodal hearing system and/or any other suitable term.

Processor 114 may be additionally or alternatively configured to receive and process data generated by cochlear implant 102. For example, processor 114 may receive data representative of a signal recorded by cochlear implant 102 using one or more electrodes 106 and, based on the data, adjust one or more operating parameters of processing unit 108. Additionally or alternatively, processor 114 may use the data to perform one or more diagnostic operations with respect to cochlear implant 102 and/or the recipient.

Other operations may be performed by processor 114 as may serve a particular implementation. In the description provided herein, any references to operations performed by processing unit 108 and/or any implementation thereof may be understood to be performed by processor 114 based on instructions 116 stored in memory 112.

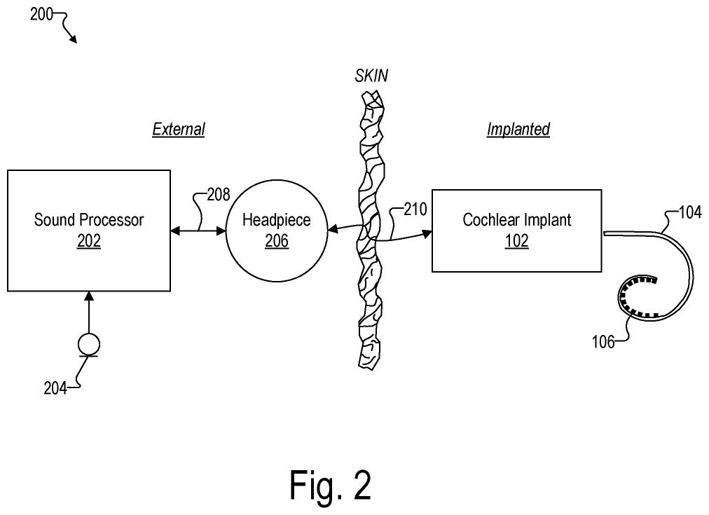

Processing unit 108 may be implemented by one or more devices configured to interface with cochlear implant 102. To illustrate, FIG. 2 shows an exemplary configuration 200 of cochlear implant system 100 in which processing unit 108 is implemented by a sound processor 202 configured to be located external to the recipient. In configuration 200, sound processor 202 is communicatively coupled to a microphone 204 and to a headpiece 206 that are both configured to be located external to the recipient.

Sound processor 202 may be implemented by any suitable device that may be worn or carried by the recipient. For example, sound processor 202 may be implemented by a behind-the-ear (“BTE”) unit configured to be worn behind and/or on top of an ear of the recipient. Additionally or alternatively, sound processor 202 may be implemented by an off-the-ear unit (also referred to as a body worn device) configured to be worn or carried by the recipient away from the ear. Additionally or alternatively, at least a portion of sound processor 202 is implemented by circuitry within headpiece 206.

Microphone 204 is configured to detect one or more audio signals (e.g., that include speech and/or any other type of sound) in an environment of the recipient. Microphone 204 may be implemented in any suitable manner. For example, microphone 204 may be implemented by a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 202. Additionally or alternatively, microphone 204 may be implemented by one or more microphones in or on headpiece 206, one or more microphones in or on a housing of sound processor 202, one or more beam-forming microphones, and/or any other suitable microphone as may serve a particular implementation.

Headpiece 206 may be selectively and communicatively coupled to sound processor 202 by way of a communication link 208 (e.g., a cable or any other suitable wired or wireless communication link), which may be implemented in any suitable manner. Headpiece 206 may include an external antenna (e.g., a coil and/or one or more wireless communication components) configured to facilitate selective wireless coupling of sound processor 202 to cochlear implant 102. Headpiece 206 may additionally or alternatively be used to selectively and wirelessly couple any other external device to cochlear implant 102. To this end, headpiece 206 may be configured to be affixed to the recipient's head and positioned such that the external antenna housed within headpiece 206 is communicatively coupled to a corresponding implantable antenna (which may also be implemented by a coil and/or one or more wireless communication components) included within or otherwise connected to cochlear implant 102. In this manner, stimulation parameters and/or power signals may be wirelessly and transcutaneously transmitted between sound processor 202 and cochlear implant 102 by way of a wireless communication link 210.

In configuration 200, sound processor 202 may receive an audio signal detected by microphone 204 by receiving a signal (e.g., an electrical signal) representative of the audio signal from microphone 204. Sound processor 202 may additionally or alternatively receive the audio signal by way of any other suitable interface as described herein. Sound processor 202 may process the audio signal in any of the ways described herein and transmit, by way of headpiece 206, stimulation parameters to cochlear implant 102 to direct cochlear implant 102 to apply electrical stimulation representative of the audio signal to the recipient.

In an alternative configuration, sound processor 202 may be implanted within the recipient instead of being located external to the recipient. In this alternative configuration, which may be referred to as a fully implantable configuration of cochlear implant system 100, sound processor 202 and cochlear implant 102 may be combined into a single device or implemented as separate devices configured to communicate one with another by way of a wired and/or wireless communication link. In a fully implantable implementation of cochlear implant system 100, headpiece 206 may not be included and microphone 204 may be implemented by one or more microphones implanted within the recipient, located within an ear canal of the recipient, and/or external to the recipient.

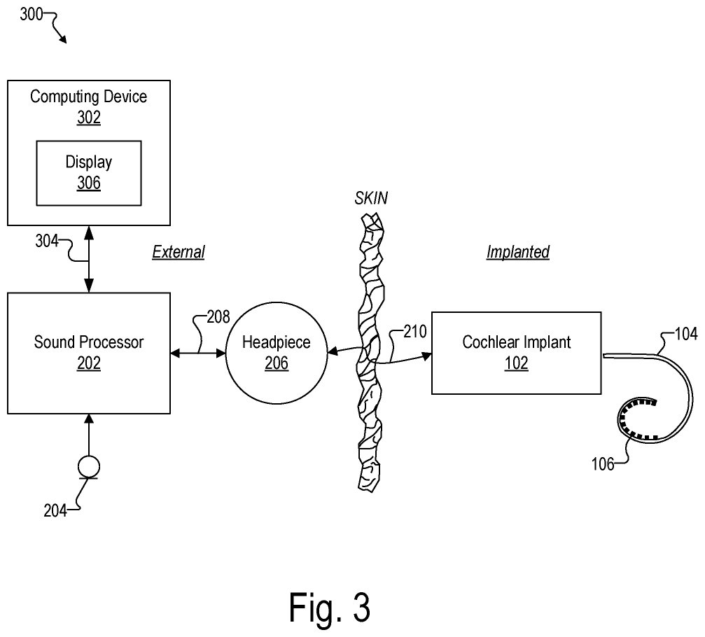

FIG. 3 shows an exemplary configuration 300 of cochlear implant system 100 in which processing unit 108 is implemented by a combination of sound processor 202 and a computing device 302 configured to communicatively couple to sound processor 202 by way of a communication link 304, which may be implemented by any suitable wired or wireless communication link.

Computing device 302 may be implemented by any suitable combination of hardware and software. To illustrate, computing device 302 may be implemented by a mobile device (e.g., a mobile phone, a laptop, a tablet computer, etc.), a desktop computer, and/or any other suitable computing device as may serve a particular implementation. As an example, computing device 302 may be implemented by a mobile device configured to execute an application (e.g., a “mobile app”) that may be used by a user (e.g., the recipient, a clinician, and/or any other user) to control one or more settings of sound processor 202 and/or cochlear implant 102 and/or perform one or more operations (e.g., diagnostic operations) with respect to data generated by sound processor 202 and/or cochlear implant 102.

In some examples, computing device 302 may be configured to control an operation of cochlear implant 102 by transmitting one or more commands to cochlear implant 102 by way of sound processor 202. Likewise, computing device 302 may be configured to receive data generated by cochlear implant 102 by way of sound processor 202. Alternatively, computing device 302 may interface with (e.g., control and/or receive data from) cochlear implant 102 directly by way of a wireless communication link between computing device 302 and cochlear implant 102. In some implementations in which computing device 302 interfaces directly with cochlear implant 102, sound processor 202 may or may not be included in cochlear implant system 100.

Computing device 302 is shown as having an integrated display 306. Display 306 may be implemented by a display screen, for example, and may be configured to display content generated by computing device 302. Additionally or alternatively, computing device 302 may be communicatively coupled to an external display device (not shown) configured to display the content generated by computing device 302.

In some examples, computing device 302 represents a fitting device configured to be selectively used (e.g., by a clinician) to fit sound processor 202 and/or cochlear implant 102 to the recipient. In these examples, computing device 302 may be configured to execute a fitting program configured to set one or more operating parameters of sound processor 202 and/or cochlear implant 102 to values that are optimized for the recipient. As such, in these examples, computing device 302 may not be considered to be part of cochlear implant system 100. Instead, computing device 302 may be considered to be separate from cochlear implant system 100 such that computing device 302 may be selectively coupled to cochlear implant system 100 when it is desired to fit sound processor 202 and/or cochlear implant 102 to the recipient.

An elastomeric encapsulant is provided as part of a cochlear implant to protect certain components of a cochlear implant system while such components are implanted within a recipient. For example, an elastomeric encapsulant may encapsulate cochlear implant 102, electrode lead 104, and/or any other suitable component.

Elastomeric encapsulants such as those described herein may be formed in any suitable manner as may serve a particular implementation. In certain examples, an elastomeric encapsulant may be overmolded around certain components (e.g., cochlear implant 102, electrode lead 104, etc.) of cochlear implant system 100. Alternatively, an elastomeric encapsulant may be formed through casting, spraying, dipping, or any other suitable manufacturing method.

In conventional electrode leads, an elastomeric encapsulant includes openings where metallic electrode contacts are conductively exposed to tissue and/or biofluid while the electrode lead is implanted within a recipient. As a consequence, an interface between the metallic electrode contacts and the elastomeric encapsulant is exposed to harsh conditions and is a point of weakness in conventional electrode leads where leakage may occur. In contrast, electrode leads such as those described herein include an elastomeric encapsulant that fully encapsulates a plurality of metallic electrode contacts and that defines an outer surface of the electrode lead. Instead of having openings that expose metal at the electrode contacts to tissue and/or biofluid, elastomeric encapsulants such as those described herein entirely cover the electrode lead with elastomeric material. For example, the elastomeric encapsulant may include a non-conductive portion configured to define a portion of the outer surface of the electrode lead and a plurality of conductive portions that form a remainder of the outer surface of the electrode lead.

The plurality of metallic electrode contacts may be formed of any suitable metal or combination of metals. For example, the plurality of metallic electrode contacts may be formed of titanium, platinum, and/or gold. In certain examples, the type of metal used for the plurality of metallic electrode contacts may be selected based on adhesion properties between the metal and the conductive portion of an elastomeric encapsulant. For example, gold electrode contacts bond much better with sulfur-containing elastomers than other elastomers. Titanium electrode contacts, on the other hand, are highly compatible with silane adhesion promoters.

The plurality of conductive portions of an elastomeric encapsulant may be formed of any suitable biocompatible conductive material. For example, the conductive portions may be formed of a conductive elastomer or of silicone that is impregnated with conductive particles (e.g., graphite powder, carbon particles, platinum particles, and/or titanium particles), conductive droplets (e.g., liquid metals such as eutectic GaIn alloys, conductive polymers, or other intrinsically conductive inks such as graphene and MXene), conductive fibers, and/or other geometries to render them conductive. Each of the conductive portions included in the plurality of conductive portions is in conductive contact with a respective metallic electrode contact included in the plurality of metallic electrode contacts and is configured to provide a current path for electrical stimulation and/or a current path to ground. Examples of conductive portions of an elastomeric encapsulant are described herein.

In certain examples, the conductive material impregnated within some of the conductive portions included in the plurality of conductive portions may be different than the conductive material impregnated within other conductive portions included in the plurality of conductive portions. For example, a first conductive portion may be impregnated with a first amount of conductive particles and a second conductive portion may be impregnated with a second amount of conductive particles that is different than the first amount of conductive particles. For example, a more distally located conductive portion may be impregnated with a relatively larger amount of conductive particles than a more proximally located conductive portion. Alternatively, a more distally located conductive portion may be impregnated with a relatively lower amount of conductive particles than a more proximally located conductive portion. In certain examples, the amount of conductive particles impregnated within the conductive portions may gradually increase toward a distal end of an electrode lead. Such a configuration may be beneficial in implementations where electric field steering is desirable.

The non-conductive portion of an elastomeric encapsulant may be considered to be any portion of the elastomeric encapsulant of the electrode lead that does not include the plurality of conductive portions. In other words, the non-conductive portion is any portion that is not in conductive contact with a metallic electrode contact of an electrode lead. The non-conductive portion of an elastomeric encapsulant may be formed of any suitable biocompatible insulative material. For example, the non-conductive portion of the elastomeric encapsulant may be formed of medical grade silicone, polyurethane, a thermoplastic elastomer, and/or any other suitable material including composite blends of these materials. The non-conductive portion of the elastomeric encapsulant is configured to bond to an outer periphery of the conductive portions of the elastomeric encapsulant during manufacture such that there is no void, opening, or other significant discontinuity on an exterior surface of the encapsulant that separates the conductive portion and the non-conductive portions where biofilms could form and/or where leakage could occur. Examples of non-conductive portions of an elastomeric encapsulant are described herein.

In certain examples, cochlear implant 102 may be encapsulated with a conductive portion of an elastomeric encapsulant. Additionally or alternatively, a fantail region (e.g., a region connecting cochlear implant 102 to electrode lead 104) may be encapsulated with a non-conductive portion of the elastomeric encapsulant. With such a configuration, it may be possible to achieve lower ground impedance while improving durability (e.g., impact imunity) of the implanted components.

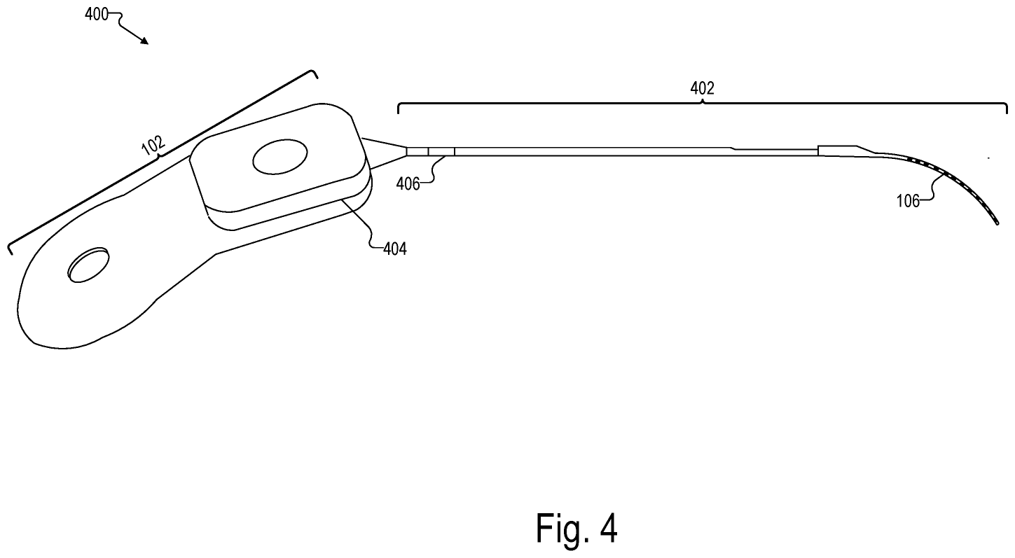

FIG. 4 illustrates an exemplary cochlear implant assembly 400 that is adapted for insertion into a recipient. As shown in FIG. 4, cochlear implant assembly 400 includes an electrode lead 402 that is communicatively coupled to an implantable cochlear implant stimulator (“ICS”) 404 that is included as part of cochlear implant 102. ICS 404 may be configured to apply electrical stimulation to a recipient by way of electrode lead 402. Electrode lead 402 includes a plurality of metallic electrode contacts including electrodes 106 and a ground electrode 406. Ground electrode 406 may be configured to provide a current return path for stimulation current applied by electrodes 106 and may also be used during perioperative impedance measurements. The configuration of ground electrode 406 shown in FIG. 4 is provided for illustrative purposes only. It is understood that a ground electrode may be configured differently than that shown in FIG. 4 in different implementations. For example, in certain alternative implementations, a ground electrode may be implemented as a conductive elastomer bar or line provided toward a distal end of an electrode lead. In such examples, the conductive elastomer bar or line may extend as far distally as at least some of the metallic electrode contacts embedded within in the electrode lead. With such a configuration, it may be possible to provide a return electrode that is much closer to the stimulating contacts of the electrode lead than ground electrode 406 shown in FIG. 4 or a case ground without significantly changing the mechanical properties of the electrode array.

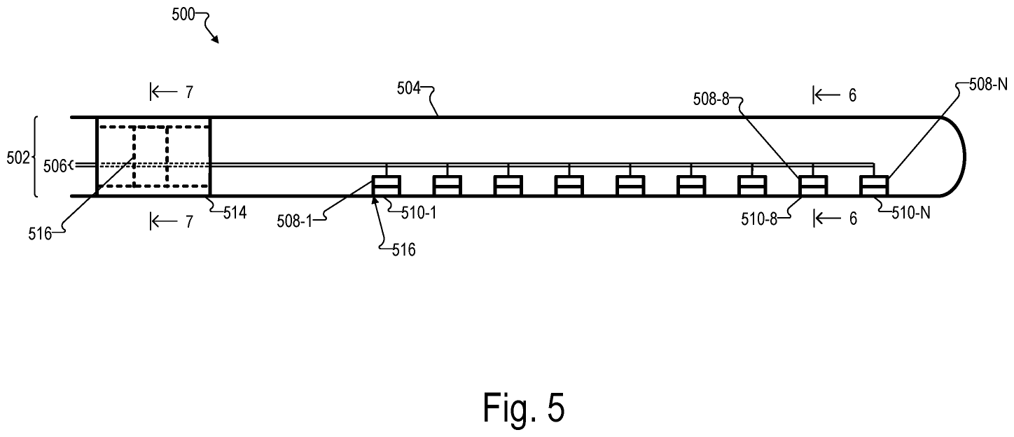

FIG. 5 shows a side view of an exemplary electrode lead 500 that may be implemented in certain examples as part of a cochlear implant assembly such as that shown in FIG. 4. As shown in FIG. 5, electrode lead 500 includes an elastomeric encapsulant 502 that includes a non-conductive portion 504 that encapsulates a plurality of wires 506 that are embedded within non-conductive portion 504 and that are configured to electrically connect a plurality of metallic electrode contacts 508 (e.g., metallic electrode contacts 508-b through 508-N) to at least one signal source (e.g., ICS 404).

Elastomeric encapsulant 502 shown in FIG. 5 further includes a plurality of conductive portions 510 (e.g., conductive portions 510-1 through 510-N) that are positioned so as to be in conductive contact with a respective metallic electrode contact 508. For example, metallic electrode contact 508-1 is in conductive contact with conductive portion 510-1, metallic electrode contact 508-8 is in conductive contact with conductive portion 510-8, and so forth.

In the example shown in FIG. 5, metallic electrode contacts 508 are depicted as having the same width as the corresponding conductive portions 510. However, it is understood that in certain examples a metallic electrode contact 508 and a corresponding conductive portion may have a different width when viewed in a side view such as that shown in FIG. 5. For example, metallic electrode contact 508-1 may have a relatively smaller width than conductive portion 510-1 when viewed in a side view such as that shown in FIG. 5. Alternatively, metallic electrode contact 508-1 may have a relatively larger width than conductive portion 510-1 when viewed in a side view such as that shown in FIG. 5.

In the example shown in FIG. 5, the plurality of conductive portions 510 and the adjacent portions of non-conductive portion 504 form a continuous coplanar outer surface of elastomeric encapsulant 502. With such a configuration, there is no void or opening in elastomeric encapsulant 502 where metallic electrode contacts 508 would be exposed to tissue and/or biofluid. In addition, the outer surface of elastomeric encapsulant 502 includes elastomer/elastomer interfaces instead of metal/elastomer interfaces. For example, an elastomer/elastomer interface 512 shown in FIG. 5 corresponds to an interface between non-conductive portion 504 and conductive portion 510-1 of elastomeric encapsulant 502. The elastomer/elastomer interfaces between, for example, non-conductive portion 504 and conductive portions 510 are configured to hermetically seal electrode lead 500 to prevent moisture from leaking into electrode lead 500 through elastomer/elastomer interfaces such as elastomer/elastomer interface 512. Such a hermetic seal may result from a non-conductive portion bonding to the conductive portion at the elastomer/elastomer interface. The bond may correspond to any suitable type of bond as may serve a particular implementation. For example, in certain implementations, a chemical bond (e.g., silicone to silicone impregnated with conductive particles) may seal the elastomer/elastomer interfaces. In certain examples, such a chemical bond may result from direct reaction of uncured elastomers. Alternatively, plasma treatments and/or any other suitable chemical bonding methods (e.g., using silane-based adhesion promoters) may be used to bond conductive portions 510 to non-conductive portion 504 at the elastomer/elastomer interfaces.

Elastomeric encapsulant 502 depicted in FIG. 5 further includes a conductive portion 514 that is in conductive contact with a metallic electrode contact 516. The combination of conductive portion 514 and metallic electrode contact 516 may function as a ground electrode or an IE and may form a current path to ground for electrode lead 500. By using a conductive elastomer for conductive portion 514 it is possible to provide a relatively larger IE region than is provided in conventional electrode leads. This relatively larger IE region may reduce the impact of ground impedance on a cochlear implant system. In addition, the relatively larger size of the IE region may increase the tissue contact surface area, which may reduce the impedance at the ground. Such a configuration may further reduce error during perioperative impedance measurements. Moreover, using a conductive elastomer as conductive portion 514 instead of a rigid (e.g., metallic) electrode reduces a mechanical mismatch and, therefore, strain at the interface between conductive portion 514 and non-conductive portion 504.

In certain examples, conductive portion 514 may have a contact surface that is configured to flexibly conform to a surface of tissue of a recipient. As shown in FIG. 5, conductive portion 514 is relatively wider than metallic electrode contact 516. As a result, a lower outer surface of conductive portion 514 depicted in FIG. 5 may be configured to flexibly conform to a surface of tissue. In such examples, the portion of electrode lead 500 that includes at least conductive portion 514 may bend to follow a surface contour of tissue while inserted within a recipient. With such a configuration, conductive portion 514 may be better suited to contact tissue than a relatively more narrow and shorter metal IE ring of a conventional electrode lead and a relatively more wide metal IE ring that would be rigid and thus not configured to flexibly conform to a surface of tissue.

In the example shown in FIG. 5, each of conductive portions 510 have a same width as metallic electrode contacts 508. For example, conductive portion 510-1 has the same width as metallic electrode contact 508-1. However, in certain alternative implementations, each metallic electrode contact may have a same width while at least some conductive portions included in a plurality of conductive portions may have different widths. For example, in certain alternative implementations, a first conductive portion included in the plurality of conductive portions may have a first width and a second conductive portion included in the plurality of conductive portions may have a second width that is different than the first width. In certain examples, the second conductive portion may be provided more distally on an electrode lead than the first conductive portion and the second width may be relatively larger than the first width. In certain alternative implementations, the second conductive portion may be provided more proximally on an electrode lead than the first conductive portion and the second width may be relatively larger than the first width. In certain examples, the widths of conductive portions on an electrode lead may progressively increase toward a distal end of the electrode lead. With such a configuration, a relatively larger electrode area on a distal end of the electrode lead may beneficially provide relatively lower impedance to low frequency stimulation regions.

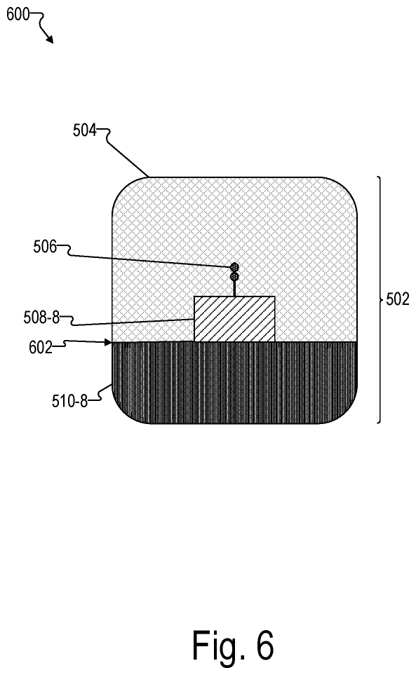

FIG. 6 depicts a cross-sectional view 600 of electrode lead 500 taken along line 6-6 in FIG. 5. In the example shown in FIG. 6, elastomeric encapsulant 502 includes non-conductive portion 504 and conductive portion 510-8. The combination of non-conductive portion 504 and conductive portion 510-8 completely surround and encapsulate plurality of wires 506 and metallic electrode contact 508-8. An elastomer/elastomer interface 602 shown in FIG. 6 corresponds to an interface between non-conductive portion 504 and conductive portion 510-8 of elastomeric encapsulant 502. The relative sizes, thicknesses, and/or shapes of the various elements depicted in FIG. 6 are provided for illustrative purposes only. For example, metallic electrode contact 508-8 may have any suitable size, thickness, and/or shape as may serve a particular implementation. In addition, it is understood that, for example, that conductive portion 510-8 may have any suitable thickness relative to metallic electrode contact 508-8. For example, in certain implementations, conductive portion 510-8 may correspond to a thin coating on a surface of metallic electrode contact 508-8. In certain examples, the cross-sectional thickness of conductive portions 510 such as conductive portion 510-8 shown in FIG. 5 may be optimized to avoid issues with compliance voltage and/or reduce power consumption.

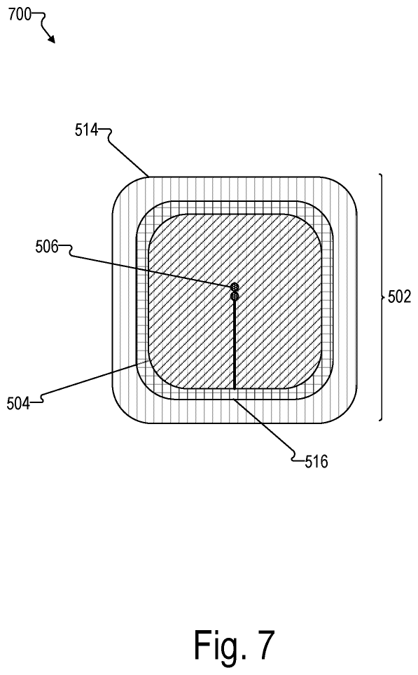

FIG. 7 depicts a cross-sectional view 700 of electrode lead 500 taken along line 7-7 in FIG. 5. As shown in FIG. 7, conductive portion 514 has a ring-like configuration such that conductive portion 514 of the elastomeric encapsulant surrounds non-conductive portion 504. Metallic electrode contact 516 also has a ring-like configuration but is surrounded by conductive portion 514 such that conductive portion 514 is not exposed at the outer surface of elastomeric encapsulant 502. As such, electrode lead 500 implements a conductive elastomeric IE ring for a ground electrode instead of an exposed metallic IE ring that may be provided in conventional electrode leads.

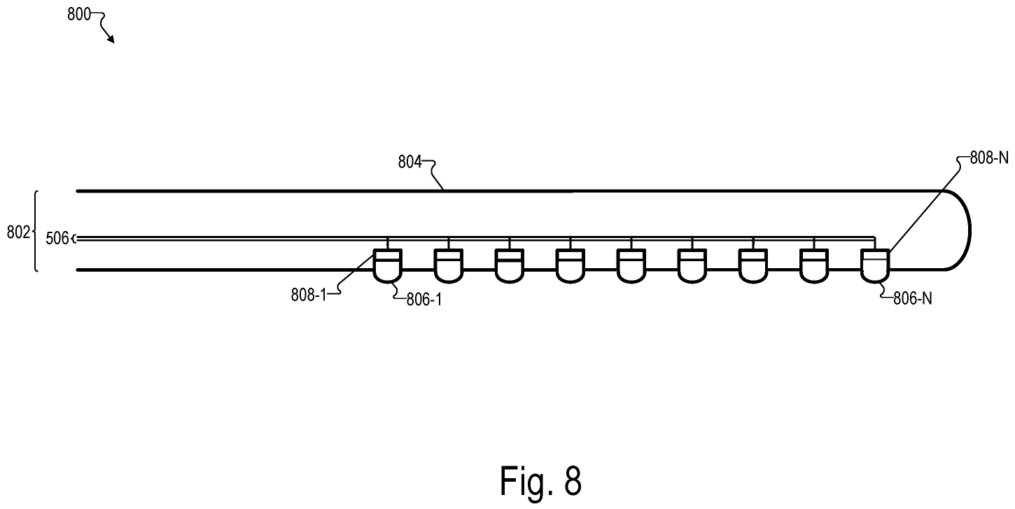

In certain examples, at least some conductive portions included in the plurality of conductive portions of an elastomeric encapsulant may protrude with respect to a non-conductive portion of the elastomeric encapsulant. To illustrate, FIG. 8 shows an exemplary electrode lead 800 that includes an elastomeric encapsulant 802 having a non-conductive portion 804 and a plurality of conductive portions 806 (e.g., conductive portions 806-1 through 806-N) that are in conductive contact with a plurality of metallic electrode contacts 808 (e.g., 808-1 through 808-N). As shown in FIG. 8, conductive portions 806 protrude with respect to adjacent sections of non-conductive portion 804 on an outer surface of elastomeric encapsulant 802. Such a protruding configuration may facilitate closer contact to neurons and/or may increase a contact surface area of electrode lead 800, which may reduce stimulation thresholds and/or impedance.

In the example shown in FIG. 8, each of conductive portions 806 protrude with respect to non-conductive portion 804 when viewed from a side of electrode lead 800. However, it is understood that, in certain alternative examples, some of the conductive portions may protrude with respect to the non-conductive portion while other of the conductive portions may form a coplanar continuous outer surface with the non-conductive portion. For example, a first set of conductive portions on a distal end of an electrode lead may protrude with respect to the non-conductive portion and a second set of conductive portions positioned towards a proximal end of the electrode lead may form a coplanar continuous outer surface with the non-conductive portion. Alternatively, a first set of conductive portions on a distal end of an electrode lead may form a coplanar continuous outer surface with the non-conductive portion and a second set of conductive portions positioned towards a proximal end of the electrode lead may protrude with respect to the non-conductive portion.

In certain examples, a plurality of metallic feedthrough pillars (not shown) may be provided within an electrode lead to conductively connect wires (e.g., wires 506) within the electrode lead to the plurality of metallic electrode contacts (e.g., metallic electrode contacts 508). In certain alternative examples, conductive elastomer traces and/or a plurality of conductive elastomer feedthrough connectors may be provided within the electrode lead instead of the wires and/or the plurality of metallic feedthrough pillars. With such a configuration, the electrode lead may be relatively more flexible and subject to relatively less strain than conventional electrode leads. In addition, with such a configuration it may be possible to avoid a tedious process of manually crimping contact wires to metallic feedthrough pillars during manufacture of an electrode lead.

Such conductive elastomer traces and/or the plurality of conductive elastomer feedthrough connectors may be formed in any suitable manner. For example, such conductive elastomer traces and/or the plurality of conductive elastomer feedthrough connectors may be formed by a three-dimensional (“3D”) printing process in certain implementations.

In certain examples, a non-conductive portion and/or a plurality of conductive portions may be impregnated with a hydrophobic material configured to reduce a rate of moisture absorption into an electrode lead. Any suitable type of hydrophobic material may be used to reduce the rate of moisture absorption as may serve a particular implementation. For example, the hydrophobic material may comprise carbon black and/or any other suitable hydrophobic material. To illustrate an example, liquid silicone rubber (“LSR”) 70 impregnated with carbon black particles may exhibit increased hydrophobicity as compared to pure LSR 70. Additionally or alternatively, an outer surface of an electrode lead may be subject to a fluorination process to increase the hydrophobicity of the outer surface. In certain examples, the hydrophobic material may also provide an elastomeric encapsulant with increased resistance to crack propagation, which is one of the major ways that an elastomer such as silicone may fail after absorbing moisture. In such examples, the hydrophobic material may act as a physical barrier to crack propagation through the elastomeric encapsulant.

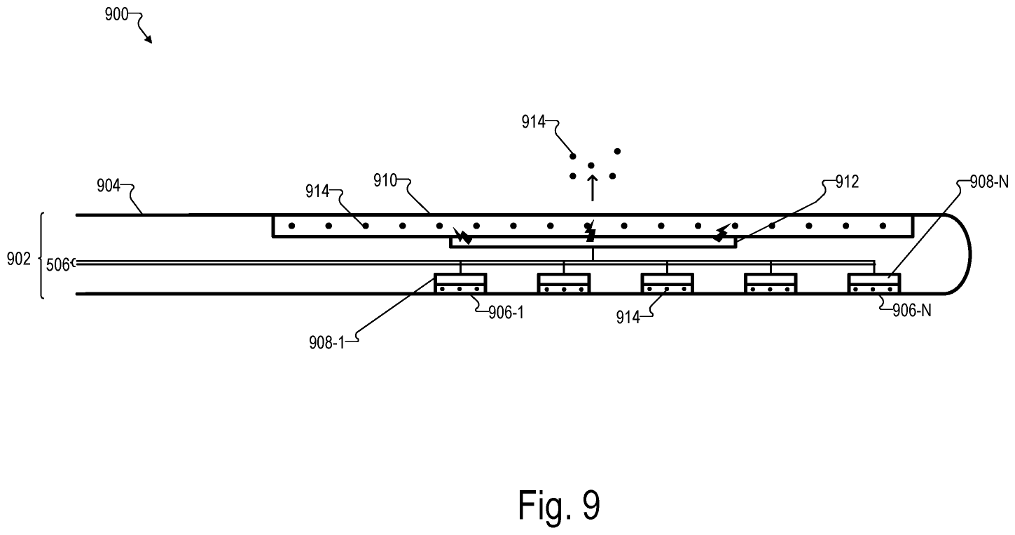

In certain examples, a conductive portion included in a plurality of conductive portions of an elastomeric encapsulant may be impregnated with a pharmaceutical. Such a pharmaceutical may be configured to be released from the elastomeric encapsulant in any suitable manner. For example, the increased hydrophobicity (e.g., due to a fluorination process) may be used to control release kinetics of a compound such as a pharmaceutical impregnated into the conductive portion. Additionally or alternatively, a pharmaceutical may be controllably released from the conductive portion and into the recipient upon application of any suitable electrical waveforms to the conductive portion. The pharmaceutical may correspond to any suitable pharmaceutical or combination of pharmaceuticals that may be controllably released into a recipient. For example, the pharmaceutical may correspond to dexamethasone in certain examples. To illustrate, FIG. 9 shows an exemplary configuration of an electrode lead 900 that includes an elastomeric encapsulant 902 comprising a non-conductive portion 904 and a plurality of conductive portions that include conductive portions 906 (e.g., conductive portions 906-1 through 906-N) and a conductive portion 908. A plurality of metallic electrode contacts including metallic electrode contacts 910 (e.g., metallic electrode contacts 908-1 through 908-N) and metallic electrode contact 912 are provided with respect to conductive portions 906 and conductive portion 910. In the example shown in FIG. 9, conductive portion 910 and metallic electrode contact 912 may together form a ground electrode of electrode lead 900. Any suitable electrical waveform may be provided by way of metallic electrode contact 912 into conductive portion 910 to cause the controlled release of a pharmaceutical 914 into a recipient.

Electrode leads such as those described herein may be manufactured in any suitable manner. For example, in certain implementations, a non-conductive elastomeric material such as silicone may be provided into a mold that includes metal wires and a plurality of metallic electrode contacts (or pillars). A conductive elastomeric material such as silicone impregnated with conductive particles may be provided over the metal wires, the metallic electrode contacts, and the non-conductive elastomeric material. This may be accomplished in any suitable manner. For example, in certain implementations, the providing of the elastomeric material and the conductive elastomeric material over the plurality of metallic electrode contacts may include simultaneously injecting the elastomeric material and the conductive elastomeric material into the mold that includes the metal wires and the plurality of metallic electrode contacts.

In certain alternative implementations, the conductive elastomeric material may be provided after the non-conductive elastomeric material is formed into an elastomeric housing. For example, the conductive elastomer may be coated on the metal wires and the metallic electrode contacts, the elastomeric housing and the metal wires and the metallic electrode contacts may be dipped in the conductive elastomer, or the conductive elastomer may be overmolded over the elastomeric housing, the metal wires, and the metallic electrode contacts.

In certain examples, the non-conductive elastomeric material and the conductive elastomeric material may be cured after providing them over the metal wires and the metallic electrode contacts.

After the conductive elastomer is provided over the metal wires and the metallic electrode contacts, at least some portions of the conductive elastomer may be removed to define a plurality of conductive portions. This may be accomplished in any suitable manner. For example, laser ablation may be used in certain implementations to remove conductive elastomer between electrode channels, to remove electrical shorts, and to produce a desired amount of separation between conductive portions, a desired shape, and/or a desired size of the plurality of conductive portions of an elastomeric encapsulant.

In certain examples, additional non-conductive elastomer may be provided in the recesses formed, for example, by the laser ablation. The additional non-conductive elastomer may be provided within the recesses in any suitable manner. For example, the combination of the conductive portions and the non-conductive portions may be dipped in additional non-conductive elastomer to fill in the recesses. In certain examples, the conductive portions and the non-conductive portions may be cured after filling in the recesses.

Although the preceding disclosure describes electrode leads configured for use with a cochlear implant system (e.g., cochlear implant system 100), it is understood that concepts such as those described herein may be applied to any other suitable type of implantable electrode or externally used electrode that may be implemented in any other suitable context. For example, concepts such as those described herein may be applied to electrode leads used in neuroprosthetic systems, neurostimulation systems, optical nerve stimulation systems, cardiac stimulation systems, etc.

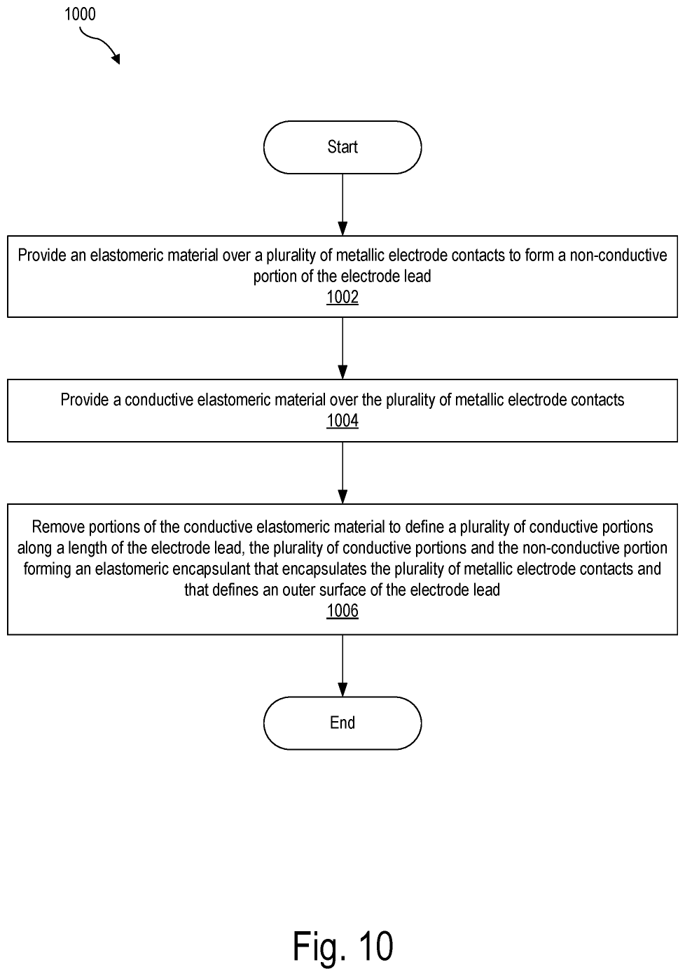

FIG. 10 illustrates an exemplary method 1000 for manufacturing an electrode lead (e.g., electrode lead 104). While FIG. 10 illustrates exemplary operations according to one embodiment, other embodiments may omit, add to, reorder, and/or modify any of the operations shown in FIG. 10.

At operation 1002, an elastomeric material may be provided over a plurality of metallic electrode contacts to form a non-conductive portion of an electrode lead. Operation 1002 may be performed in any of the ways described herein.

At operation 1004, a conductive elastomeric material may be provided over the plurality of metallic electrode contacts. Operation 1004 may be performed in any of the ways described herein.

At operation 1006, portions of the conductive elastomeric material may be removed to define a plurality of conductive portions along a length of the electrode lead. The plurality of conductive portions and the non-conductive portion may form an elastomeric encapsulant that encapsulates the plurality of metallic electrode contacts and that defines an outer surface of the electrode lead. Operation 1006 may be performed in any of the ways described herein.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A cochlear implant assembly adapted for insertion into a recipient, comprising:
an electrode lead; and
an implantable cochlear stimulator (ICS) configured to apply electrical stimulation to the recipient by way of the electrode lead,
wherein the electrode lead includes:
a plurality of metallic electrode contacts; and
an elastomeric encapsulant that encapsulates the plurality of metallic electrode contacts and that defines an outer surface of the electrode lead, the elastomeric encapsulant including:
a non-conductive portion configured to define a portion of the outer surface of the electrode lead; and
a plurality of conductive portions that form a remainder of the outer surface of the electrode lead, each of the conductive portions included in the plurality of conductive portions in conductive contact with a respective metallic electrode contact included in the plurality of metallic electrode contacts.

2. The cochlear implant assembly of claim 1, wherein the non-conductive portion and at least some conductive portions included in the plurality of conductive portions form a continuous coplanar exterior surface.

3. The cochlear implant assembly of claim 1, wherein at least some conductive portions included in the plurality of conductive portions protrude with respect to the non-conductive portion.

4. The cochlear implant assembly of claim 1, wherein a conductive portion included in the plurality of conductive portions forms a current path to ground for the electrode lead.

5. The cochlear implant assembly of claim 4, wherein the conductive portion has a contact surface that is configured to flexibly conform to a surface of tissue of the recipient.

6. The cochlear implant assembly of claim 1, wherein the plurality of conductive portions of the elastomeric encapsulant is formed of silicone impregnated with at least one of conductive particles, conductive droplets, or conductive fibers to enhance electrical conductivity of the plurality of conductive portions.

7. The cochlear implant assembly of claim 1, wherein at least one of the non-conductive portion or the plurality of conductive portions is impregnated with a hydrophobic material configured to reduce a rate of moisture absorption into the electrode lead.

8. The cochlear implant assembly of claim 1, wherein a conductive portion included in the plurality of conductive portions is impregnated with a pharmaceutical that is configured to be controllably released from the conductive portion and into the recipient upon application of electrical waveforms to the conductive portion.

9. An electrode lead comprising:
a plurality of metallic electrode contacts; and
an elastomeric encapsulant that encapsulates the plurality of metallic electrode contacts and that defines an outer surface of the electrode lead, the elastomeric encapsulant including:
a non-conductive portion configured to define a portion of the outer surface of the electrode lead; and
a plurality of conductive portions that form a remainder of the outer surface of the electrode lead, each of the conductive portions included in the plurality of conductive portions in conductive contact with a respective metallic electrode contact included in the plurality of metallic electrode contacts.

10. The electrode lead of claim 9, wherein the non-conductive portion and at least some conductive portions included in the plurality of conductive portions form a continuous coplanar exterior surface.

11. The electrode lead of claim 9, wherein at least some conductive portions included in the plurality of conductive portions protrude with respect to the non-conductive portion.

12. The electrode lead of claim 9, wherein a conductive portion included in the plurality of conductive portions forms a current path to ground for the electrode lead.

13. The electrode lead of claim 12, wherein the conductive portion has a contact surface that is configured to flexibly conform to a surface of tissue of a recipient of the electrode lead.

14. The electrode lead of claim 9, wherein a conductive portion included in the plurality of conductive portions is impregnated with a pharmaceutical that is configured to be controllably released from the conductive portion and into a recipient of the electrode lead upon application of electrical waveforms to the conductive portion.

15. The electrode lead of claim 9, wherein the plurality of conductive portions of the elastomeric encapsulant is formed of silicone impregnated with at least one of conductive particles, conductive droplets, or conductive fibers to enhance electrical conductivity of the plurality of conductive portions.

16. A method of manufacturing an electrode lead, comprising:
providing an elastomeric material over a plurality of metallic electrode contacts to form a non-conductive portion of the electrode lead;
providing a conductive elastomeric material over the plurality of metallic electrode contacts; and
removing portions of the conductive elastomeric material to define a plurality of conductive portions along a length of the electrode lead, the plurality of conductive portions and the non-conductive portion forming an elastomeric encapsulant that encapsulates the plurality of metallic electrode contacts and that defines an outer surface of the electrode lead.

17. The method of claim 16, further comprising:
providing the elastomeric material within recesses where the conductive elastomeric material was removed; and
curing the elastomeric material and the conductive elastomeric material.

18. The method of claim 16, wherein the removing of the portions of the conductive elastomeric material includes using laser ablation to define the plurality of conductive portions.

19. The method of claim 16, wherein:
the elastomeric material is formed of silicone; and
the conductive elastomeric material is formed of silicone impregnated with at least one of conductive particles, conductive droplets, or conductive fibers to enhance electrical conductivity of the plurality of conductive portions.

20. The method of claim 16, wherein the providing of the elastomeric material and the conductive elastomeric material over the plurality of metallic electrode contacts includes simultaneously injecting the elastomeric material and the conductive elastomeric material into a mold that includes the plurality of metallic electrode contacts.
